(19) United States
(12) Patent Application Publication
(10) Pub. No.: US 2008/0065176 A1
(43) Pub. Date: Mar. 13, 2008

(54) Vacuum and negative ions assisted phototheraphy device

(75) Inventors: Sean Zhang, Cupertino, CA (US); Shijie Gu, Sunnyvale, CA (US); Huiyun Ge, Santa Clara, CA (US)

Correspondence Address: Sean Zhang, 2362 Qume Dr. #F, San Jose, CA, 95131, US

(73) Assignee: Rainbow Communications, Inc.

(21) Appl. No.: 11/518,010

(22) Filed: Sep. 11, 2006

(51) Int. Cl. A61N 5/06

(52) U.S. Cl. 607/88

(57) ABSTRACT

This invention discloses an apparatus for skin treatments. The apparatus comprising a chamber placed on a skin target which is formed with an aperture on the distal end thereof; means for applying vacuum to said chamber, therefore the skin target is able to be treated upon the applied vacuum; and/or means for supplying negative ion into said chamber; and c) at least one light source with one wavelength or multi-wavelength, wherein the light source is arranged inside the chamber to provide optical energy in the predetermined wavelength(s) through the aperture to the skin target. With the assistance of negative ion as well as vacuum, the skin treatment will be improved because it purifies the air and relieves stress to enhance activation of skin cell, accelerate blood cycle.

### FIELD OF THE INVENTION

The present invention is related to the field of skin treatments. More specifically, the invention is related to the utilization of one or more sources for the non-invasive treatment of skin disorders.

### BACKGROUND OF THE INVENTION

It has been disclosed that the light source with the spectrum, such as laser diodes or light emitting diodes (LED) are currently used in aesthetic treatments by one of three known ways: a) Applying the light to the skin without applying any pressure on the treatment zone, so as not to interfere with the natural absorption properties of skin; b) Applying pressure onto the skin by means of the exit window of the treatment device in contact with the skin, thereby expelling blood from the light path within the skin and enabling better transmission of the light to a skin target in cases where the spectral lines of the treatment light source match absorption lines of the blood; and c) Applying pressure to the skin by a vacuum chamber which is transparent to light on a skin target.

U.S. Pat. Application No. 20050215987 described a method and device to treat skin disorder by an intensive light source with a vacuum chamber which is transparent to light on a skin target to apply pressure. U.S. Pat. Application No. 20060030908 disclosed a LED skin treating apparatus with different shapes to adapt different positions of the face. U.S. Pat. No. 6,662,054 disclosed a device to treat skin disorder by LED with different wavelength.

Applying a vacuum to the skin is a known prior art procedure, e.g. for the treatment of cellulites, which complements massaging the skin. Such a procedure produces a flow of lymphatic fluids so that toxic substances may be released from the tissue. As the vacuum is applied, a skin fold is formed. The skin fold is raised above the surrounding skin surface, and the movement of a handheld suction device across the raised skin performs the massage. The suction device is moved in a specific direction relative to the lymphatic vessels, to allow lymphatic fluids to flow in their natural flow direction. The lymphatic valve in each lymphatic vessel prevents the flow of lymphatic fluid in the opposite direction, if the suction device were moved incorrectly. Liquids generally accumulate if movement is not imparted to the raised skin. The massage, which is generally carried out by means of motorized or hand driven wheels or balls, draws lymphatic fluids from cellulite in the adipose subcutanous region and other deep skin areas, the depth being approximately 5-10 mm below the dermis.

U.S. Pat. No. 5,961,475 disclosed a massaging device with which negative pressure is applied to the skin together during massaging. A similar massaging device which incorporates a radio frequency (RF) source for the improvement of lymphatic flow by slightly heating the adipose tissue is described in U.S. Pat. No. 6,662,054. Some massaging systems, such as those produced by Deka and Cynosure, add a low power, continuous working (CW) light source of approximately 0.1-2 W/cm², in order to provide deep heating of the adipose tissue by approximately 1-3° C. and to enhance lymphatic circulation. The light sources associated with vacuum lymphatic massage devices are incapable of inducing blood vessel coagulation due to their low power. Also, prior art vacuum lymphatic massage devices are adapted to induce skin protrusion or to produce a skin fold by applying a vacuum.

It is an object of the present invention to provide a method and apparatus for the treatment of acne and wrinkles etc. to beauty the body by a LED Array Phototherapy operating at wavelengths from 400 nm to 1800 nm which does not damage the surface of the skin or the epidermis. It is further hope to control the depth of subcutaneous light absorption and purify the air and relieve stress to enhance activation of skin cell and accelerate blood cycle.

Other objects and advantages of the invention will become apparent as the description proceeds.

### SUMMARY OF THE INVENTION

The present invention discloses an apparatus for controlling and/or purifying the skin target of light. The apparatus comprises a chamber which is transparent to monochromatic or non-coherent light on a skin target. The chamber is a container for ion to purify the treated skin and/or a vacuum chamber to absorb the treated skin. In the treatment, a pump applies a vacuum to said vacuum chamber, whereby said skin target is drawn to the said vacuum chamber. The invention also modulates the applied ion and/or vacuum ratio to improve and/or purify the treatment of the target skin, and direct the light to said skin target.

As referred to herein, “vacuum/ion modulation” means adjustment of the vacuum level/ion density within, or of the frequency by which vacuum/ion is applied to, the chamber. By properly modulating the vacuum/ion, the blood flow rate, in a direction towards the vacuum chamber, within blood vessels at a predetermined depth below the skin surface can be controlled; the surface of the skin can be purified.

In one aspect, the light source are LED arrays with the wavelength of the light ranges from 400 to 1800 nm. The level of the applied vacuum within the vacuum chamber ranges from 0 to −125 mmHg. The density of ion ranges from 0 to 1.5 million negative ions/cm³ in the chamber. The frequency of vacuum modulation ranges from 0.1 to 200 Hz. The light is power on after a predetermined delay following application of the vacuum/ion. The predetermined delay ranges from approximately 10 msec to approximately 1 second. Vacuum/ion modulation is electronically controlled by controlling the valve.

In one embodiment of the invention, the apparatus comprises: a) a chamber placed on a skin target which is formed with an aperture on the distal end thereof b) means for supplying ion into said chamber, therefore the ion can be used to treat the skin target; and c) LED array as light source. In the apparatus, there is a control means for controlling the ion density by controlling the means for supplying ion.

In another embodiment, the apparatus comprises: a) a chamber placed on a skin target which is formed with an aperture on the distal end thereof b) means for applying vacuum to said chamber, therefore the skin target is able to be treated upon the applied vacuum; and c) at least one light source with one wavelength or multi-wavelength, wherein the light source is arranged inside the chamber to provide optical energy in the predetermined wavelength(s) through the aperture to the skin target.

In still another embodiment, the apparatus for skin treatments, comprises: a) a chamber placed on a skin target which is formed with an aperture on the distal end thereof b) means for supplying ion into said chamber, therefore the ion can be used to treat the skin target and at least one light source with one wavelength or multi-wavelength, wherein the light source is arranged inside the chamber to provide optical energy in the predetermined wavelength(s) through the aperture to the skin target.

In summary this invention disclosed a most preferred embodiment of a handhold device for treatment of the skin based on Light Emitting Diode (LED) assisted by vacuum and negative ions. Most preferred wavelength of the light ranges from 400 to 880 nm according to treatment of the skin. The LED's head of the device can be exchanged quickly and easily for different wavelengths combinations. In a LED array head, there have several tens of LEDs with same or mixed wavelengths illuminations. The intensity of device output light can be adjusted up to 20-50 mW/cm². There is a chamber for vacuum and/or negative ions before LED's head. A build-in MEMS or other mini vacuum pump supplies negative pressure (125 mmHg) in the chamber. A build-in mini negative ion generator supplies negative ion with high density (>1 million negative ions/cm³) in the chamber. And a build-in mini 3-way solenoid valve controls mode of vacuum or negative ion in the chamber;

With the invention, the treatment of skin target is improved and purified by the negative ion and/or negative pressure. In order to make the device portable, a MicroElectroMechanical System (MEMS) based pump and valve components are used for the vacuum and ion system, as well as some exchangeable light source head with several LEDs are used.

### BRIEF DESCRIPTION OF DRAWINGS

FIG. 1 is a perspective view of the preferred embodiment of a handhold phototherapy device for treatment of the skin condition based on Light Emitting Diode (LED) assisted by both vacuum and negative ion systems of the invention;

FIG. 2 is the preferred embodiment of the LED array head of the phototherapy device of FIG. 1, which shows the structure of the head and how to exchange the head;

FIG. 3 is a perspective view of the preferred embodiment of LED array head of the phototherapy device of FIG. 1;

FIG. 4 shows the principle of layout of LED in the LED array head of the embodiment of FIG. 1; wherein FIG. 4a shows the first embodiment with thirty Red LEDs in the head with the wavelength of 630 nm; FIG. 4b shows the second embodiment of the head with thirty Blue LEDs with the wavelength around 405 nm; FIG. 4c shows the third embodiment of the head with fifteen Blue LEDs and fifteen Red LEDs.

FIG. 5 shows the principle diagram of the embodiment of negative pressure and negative ions during treatment of the skin.

FIG. 6 is a schematic diagram of the mini negative ion generator in the embodiment of FIG. 1.

### DETAILED DESCRIPTION OF PREFERRED EMBODIMENT

Reference is now made to the figures in which like reference numerals refer to like elements. For clarity, the first digit of a reference numeral indicates the figure number in which the corresponding element is first used. While the various aspects of the embodiments disclosed are presented in drawings, the drawings are not necessarily drawn to scale.

Those skilled in the art will recognize that the systems and methods disclosed can be practiced without one or more of the specific details, or with other methods, components, materials, etc. In some cases, well-known structures, materials, or operations are not shown or described in detail. Furthermore, the described features, structures, or characteristics may be combined in any suitable manner in one or more embodiments. It will also be readily understood that the components of the embodiments as generally described and illustrated in the figures herein could be arranged and designed in a wide variety of different configurations.

Acne is a condition of the sebaceous glands that affects 80% of the human population. Acne usually starts in adolescence when hormonal changes cause the enlargement and then the obstruction of sebaceous glands in the skin. The obstruction of the glands' openings causes the accumulation of sebum, which is followed by abnormal proliferation of bacterial population, predominantly Propionibacterium acnes. Acnes attract inflammatory cells and thus the unsightly red, painful pustules of acne are formed. These same lesions potentially heal with permanent scarring. In spite of various available treatments for acne, there are many patients who fail to respond adequately or who develop problematic side effects. Topical acne medications are usually irritating to the skin: more than 40% of acne bacteria are insensitive to oral antibiotics and therapy with oral isotretinoin (Accutane2), which is also associated with possible severe side effects and a high cost. Sun exposure is known to be beneficial in up to 70% of patients with acne. Although solar and/or artificial ultraviolet (UV) light has a mild camouflage effect on acne, their comedogenic and photoaging effects prevent their use in acne therapy. It is known that P. acnes produce, during their normal life cycle, as a part of their normal metabolism, porphyrines; mainly coproporphyrines. Visible light in the blue range induces a photo-destructive effect on P. acnes that may take part in the decrease in acne severity in summer. Blue-violet light (405-420 nm) has been shown to be 10 times more effective than red light (630-670 nm) in triggering excitation of coproporphyrines. Red light may have anti-inflammatory properties by influencing the release of cytokines from macrophages or other cells but its exact mode of action in the treatment of acne vulgaris is not yet fully understood.

In order to treat wrinkles in the skin, blue, red and yellow wavelength bands may be used. The blue and red wavelength ranges are 400 to 470 nm and 630 to 680 nm, respectively. The yellow band of wavelengths may be between 530 and 600 nm. In treating rosacea a yellow range of wavelengths may be used between 530 and 600 nm. In treating sun spots, a yellow range of wavelengths (530 to 600 nm) may be used. For alternative forms of sun damage, a red band (630 to 680 nm) may be employed. Blue light (between 400 and 470 nm) may be used to treat and kill bacteria that may cause various forms of skin blemishes, such as acne. Inflammation may be treated by exposing affected skin to red wavelengths (630 to 680 nm) and also to infrared wavelengths, which may range from about 800 nm to about 1000 nm. Lesions in the skin may be treated by illuminating the affected area with red wavelengths (630 to 680 nm) and infrared wavelengths (800 to 1000 nm). Canker sores may also be treated by irradiating the sore to red and infrared wavelengths (630 to 680 nm and 800 to 1000, nm, respectively). Skin blemishes may be treated through exposure to red, blue and yellow wavelengths. As discussed above the wavelength ranges may be 630 to 680 nanometers for red, 400 to 470 nanometers for blue, and 530 to 600 nanometers for yellow.

For this application, the phrases “connected to” and “coupled to” refer to any form of interaction between two or more entities, including mechanical, electrical, magnetic, electromagnetic, fluid, and thermal interaction. Two components may be coupled to each other even though they are not in direct contact with each other.

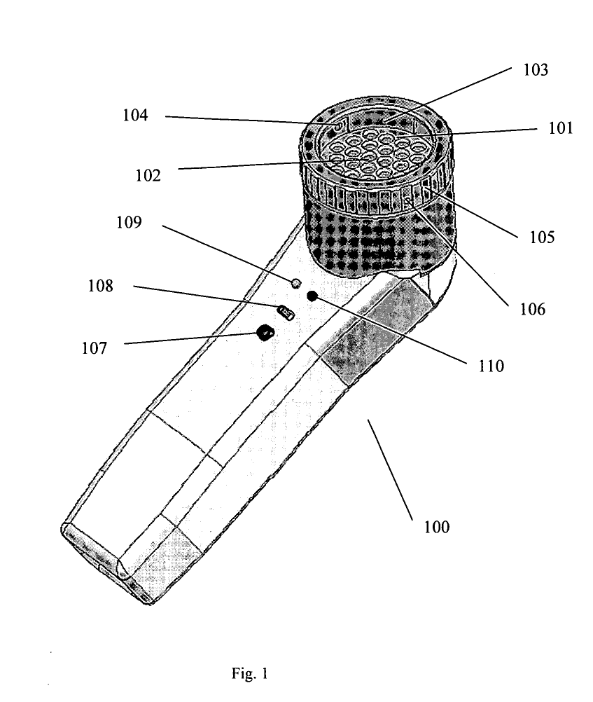

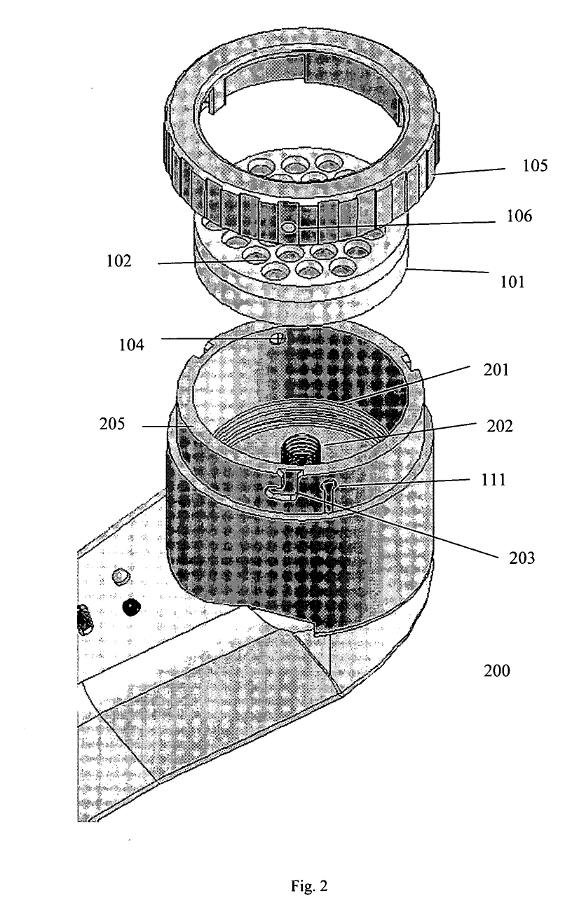

FIG. 1 is a perspective view of the preferred embodiment of a handhold phototherapy device for treatment of the skin condition based on Light Emitting Diode (LED) assisted by both vacuum and negative ions systems of the invention. The phototherapy device 100 has a LED's head 101, in which there is a LED array head 101 with several LEDs 102. In the preferred embodiment, there are thirty LEDs in the head 101. There can be more or less LEDs upon the arrangement of the array, which is well known by the skilled person in this technical field. In the embodiment, in order to treat different skin disorders as described above, the LED's head 101 can be removable from the device 100 and replaced with another color LED head or another multi-color LED head. To obtain better effect of the skin area to be treated, the head 101 is located in a chamber 103 to separate the skin to be treated from other skin. By the chamber 103, the head 101 does not directly contact the skin. In the embodiment, a vacuum port 104 is designed at the side wall of the chamber 103 for making a negative pressure when needed. To clearly understand the embodiment, now we further refer to FIG. 2, the preferred embodiment of the LED array head of the phototherapy device of FIG. 1, which shows the structure of the head and how to exchange the head. In order to provide negative ions within the chamber, a negative ion port 111 opposites to the vacuum port 104 for supplying negative ions in the chamber 103. Although the ion port 111 and vacuum port 104 are opposite in the embodiment, the skilled person in this art knows there may be many different arrangements. For locking LED array head 101 and keep the air in the chamber 103 separating from the outside air, quick ring 105 is used. In the embodiment, turning the ring 105 clockwise a few degrees, the user can lock LED head in the desired lock position by three lock pins 106 in the ring 105 which are adapted to the groove 203 in the body 205 of the device 100. Turning the ring 105 counterclockwise a few degrees, the user can take off the ring 105 and the array head 101 will jump out by the springs 201 and 202 under the head 101. Click switch 107 is a multi-function switch to select the functions of the device 100, which is one of below function or the combination thereof, turn on the power of light, turn on the vacuum pump, and turn on the negative ion generator. In the most preferred embodiment, the first click of the switch 107 is turning on the power and light, second click is turning on the vacuum, the third click is designed to turn on the negative ion generator, and click and hold the button for a couple of seconds to turn off the main power. Switch 108 is a timer select switch. Indicators 109 and 110 are LEDs to indicate battery charging and battery low, respectively. In another embodiment, there are three switches for LED power, vacuum pump power, and negative ion generator power. By these three switches, the user can first turn on the LED, and then turn on the negative ion generator, and turn on the vacuum pump. The user also can turn on the negative ion generator first to purify the skin in the chamber 103. With these three switches, the user can select the sequence of these three functions and may only use one function or two functions or three functions simultaneously.

Referring to FIG. 2 again, a preferred embodiment of the invention, two metal springs 201 and 202 in the body 205 of the chamber 103 of the device 100 are used for fixing the LED head in the device 101 as described above, these two springs 201 and 202 are also electrically coupled to the head 101 to supply power to LEDs in the LED head 101 from the battery power source (not show in these two drawings) of the device 100, wherein the small one 202 is the positive pole and the large one is the negative pole of the power. There are many different ways to lock the head 101 and to supply the power to the LEDs which are well known by the skilled person in this area, which shall be within the protection scope of the invention.

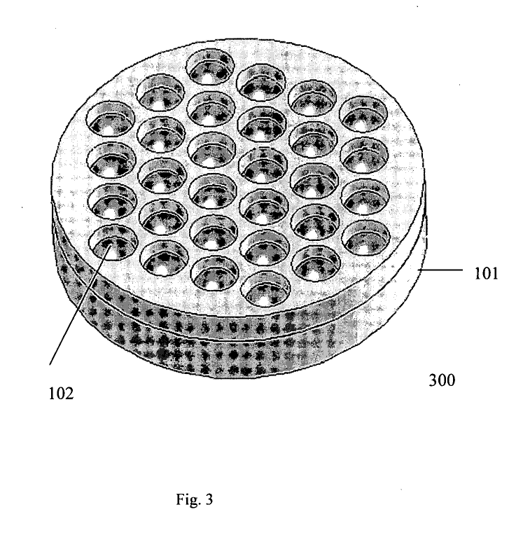

FIG. 3 is a perspective view of the preferred embodiment of LED array head 101 of the phototherapy device 100 of FIG. 1. There are many LED 102 in the arranged hole, all the LEDs 102 are coupled to the power pole of the heads pole (not show) as described by referred to FIG. 2. The arrangement of the LED array is dependent on the treatment requirement, e.g. the skin position to be treated, the shape of the skin, as nose etc. The number and the distribution of the LEDs 102 in the head 101 can be varied upon the applications. The LED array head 101 is separated from the adjoining skin surface by a gap ranging from 0.1 to 50 mm.

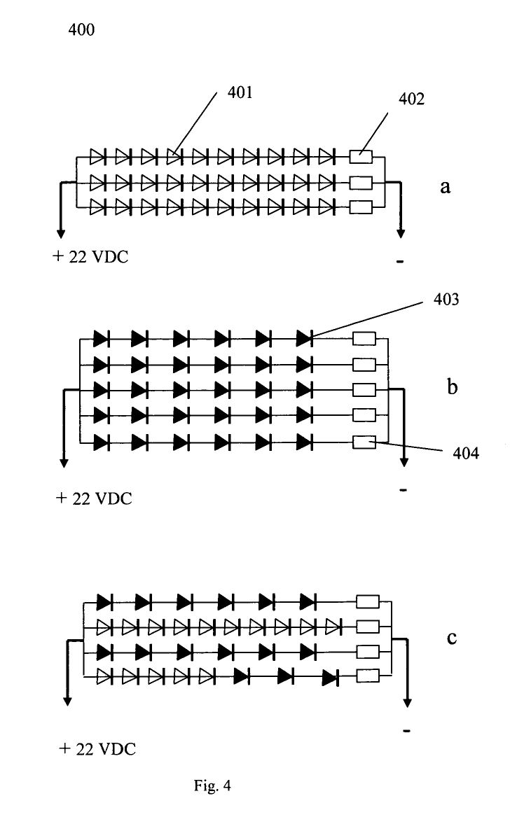

FIG. 4 shows the principle of layout of LED in the LED array head of the embodiment of FIG. 1; wherein FIG. 4a shows the first embodiment with thirty Red LEDs in the head with the wavelength of 630 nm; FIG. 4b shows the second embodiment of the head with thirty Blue LEDs with the wavelength around 405 nm; FIG. 4c shows the third embodiment of the head with fifteen Blue LEDs and fifteen Red LEDs. In the preferred embodiment of the invention, there are thirty LEDs in the LED head 101. It is known by the skilled person in the art there might be any number of LEDs which can be included in the LED's head 101. In order to exchange the LED heads with each other, one must combine LEDs with different wavelengths (color) and resistor 402 to fit the same voltage supply. Referring to FIG. 4a first, in this embodiment, there are thirty red LEDs 401, their output wavelength is 630 nm, the typical voltage applied to the LED is 2.2 VDC@20 mA current for each LED. The power supply has a voltage of 22 VDC, and the skilled person in the art can adjust the resister to adjust the voltage and current of Red LED's head. In the embodiment of FIG. 4b, there are thirty Blue LEDs 403, the output wavelength is around 405 nm, the typical voltage applied to the LED is 3.5 VDC@20 mA current for each blue LED element. The power supply has a voltage of 22 VDC, and the skilled person in the art can adjust the resister to adjust the voltage and current of Blue LED's head. In this embodiment, a set of resistors 404 of 50 ohms are used. For the embodiment showing in FIG. 4c, there are fifteen Blue LEDs and fifteen Red LEDs. The power supply has a voltage of 22 VDC, and the skilled person in the art can adjust the resister and the LED arrangement to adjust the voltage and current of this multi-color LED head.

In another embodiment, there are fifteen Blue LEDs and fifteen Red LEDs and fifteen Yellow LEDs in the LED array head 101. All these forty five LEDs are controlled by a control unit with a CPU, as 80C31 manufactured by Intel Corporation, and some memories for storing program and data. The control unit is able to power on or off any one of the forty five LEDs upon a preset model set or a programble model, so that the emitting light can be adjusted upon the requirement of the user. In one embodiment, there are seven preset models, i.e. RED, BLUE, YELLOW, RED+BLUE, RED+YELLOW, BLUE+YELLOW, and RED+BLUE+YELLOW which can set by the control panel of the device 100. In still another embodiment, the model is programble by a computer, there is an interface, e.g. USB, in the device 100. In order to set the model, one can program by a computer and input the set model via the USB to the program memory of the device 100. With such programble model, the user can adjust the model upon the development of the skin treat test in future. For example, the user may program the model as turn on ten blue LEDs 1 minute and then turn on fifteen red LEDs for 2 minutes and then turn on eight yellow LEDs and turn off five blue LEDs which was turned on for 5 minutes. Although describe upon the embodiment, the skilled person in the art knows there are many models can be set.

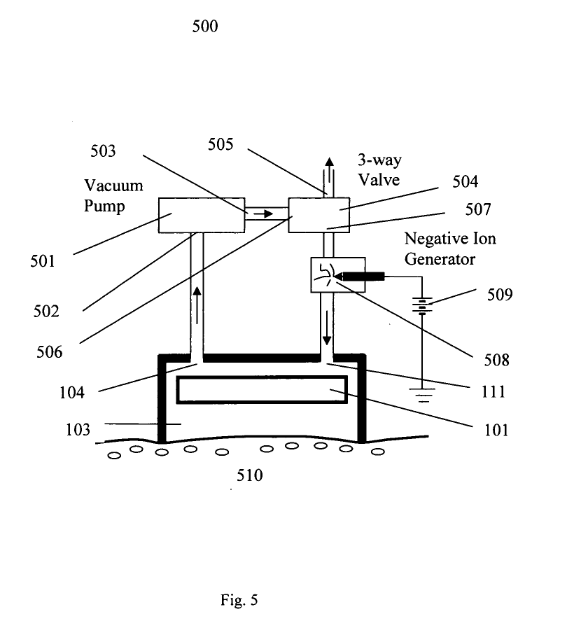

FIG. 5 shows the principle diagram of the embodiment of negative pressure as vacuum and/or negative ions during treatment of the skin. In this embodiment, to simplify the structure of the device 100 to have it is portable, negative pressure as vacuum and negative ions is only able to be supplied respectively, i.e. at one time, either vacuum or negative ion is able to be provided. After reading this description, the skilled person shall know it is easy to make the device which can applies both negative pressure and negative ions to the chamber 103. In the embodiment the wall of the chamber 103 is contacted with the treated skin target 510 to airproof for the chamber 103. In the application, in order to applied negative pressure in the chamber, the user can turn on the vacuum pump 501, the vacuum pump 501 pump air from the chamber 103 via port 104 and vacuum port 502. The pumped air is compressed and out put via an output port 503 to a 3-way valve 504. Valve output port 505 of the 3-way valve 504 is open, and port 507 is close to have the compressed air output the chamber for applying the vacuum to the chamber. In this way, the compressed air from port 503 will be sent out to the atmosphere through common ports 506 and port 505. In the embodiment, MEMS (MicroElectroMechanical System) or other mini pump 501 is used which is very small in size and light in weight, such as, Koge Electronics vacuum pump KPV14A-3A, the size is 40×12×27 mm³ (L×H×W), maximum vacuum is <−200 mmHg@100 cc volume, minimum pressure air flow is 1.5 LPM. 3-way valve 504 is similar to Lee Company LHDA0531115H mini valve, the size is 30×Ø7 mm². In other embodiment, in order to supplying negative ions into the chamber, user can turn on vacuum pump 501, port 505 of 3-way valve 504 is close and port 507 is open, so that the air is cycled from the chamber 103 to the pump 501 and to the valve 504 through the negative ion generator 508 and return to the chamber 103, i.e. the compressed air flow from port 503 through common port 506 and port 507 into the negative ion generator, the compressed air flow with a large number of negative ions pass through port 111 into the chamber 103. The port 104 sucks back the air in the chamber 103 into negative ion generator, the air is cycled between the negative ion generator and the chamber, which will make the density of negative ions in the chamber higher and higher. Even the rate of negative ion generator is only 100,000 ions/second, the density of negative ions in the chamber can be higher than a million ions/cm in minute, because the volume of the chamber is only a few cc. In the embodiment, the high voltage power supply of the negative ion generator 509 supplies a minus 3 kVDC voltage to discharge point 508. 16

In order to provide both vacuum and negative ion at same time, the invention can use two pumps, one for vacuum and one for supply negative ion as described above. Furthermore, in order to modulate the Ion/Vacuum ratio in the chamber, the pump can adjust its pumped air and the ion generator may adjust switch voltage to the discharge point.

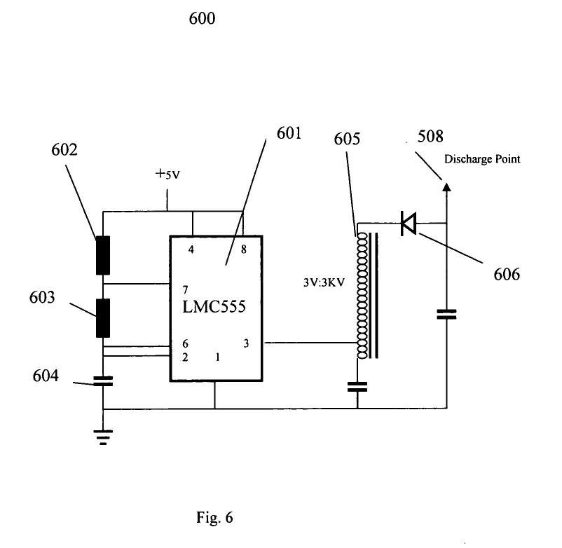

FIG. 6 is a schematic diagram of the mini negative ion generator in the embodiment of FIG. 1. Mini negative ion generator 600 transforms 5 VDC of rechargeable batteries to higher than 3 kVDC voltage, which supplies discharge point 508 to produce negative ions in the air. The timer 601 (LMC555) transforms direct current signal into square-wave pulse signal with high frequency (100 kHz). High frequency transformer 605 transforms low voltage high frequency pulse signal into high voltage high frequency pulse signal. High voltage rectifier diode 606 (NTE518) transforms high voltage high frequency pulse signal into high voltage direct current signal. Resistors 602 and 603 are 1000 and 300 Ω, respectively. Capacitor 604 is 9 nf, which makes pulse frequency 100 kHz. In this design one does not use Darlington transistor for large current output, the timer output current itself can generate enough negative ions for this application.

By the assistance of negative pressure and/or negative ion, the invented light therapy device works in conjunction with high-density negative ions to purify the air and relieve stress, which can aggravate skin and the acne conditions. Some test shows that negative ions can enhance activation of skin cell, accelerate blood cycle. Most clinicians use Topical Negative Pressure (TNP) therapy at a sub-atmospheric pressure of 125 mm Hg—that is, 125 mm Hg below atmospheric pressure. Evidence for this being the optimum pressure is limited. The test using laser Doppler needle flow robes in the acute wound swine model showed that local tissue perfusion increased with decreasing pressures up to 125 mm Hg below atmospheric pressure. Although, further decreases in pressure led to a decrease in local tissue perfusion giving a U shaped distribution. TNP therapy increases blood flow, decreases bacterial colonization and stimulates the growth of granulation tissue of skin.

In the invented device, MicroElectroMechanical System (MEMS) based pump(s) and valve(s) are used for the vacuum unit, which will make the device more rugged, compact, and reliable as well as cheaper. Other mini vacuum pumps are also good depending on the size and weight requirements.

In order to treat different skin condition and also simplify the device, the invention is of Head Exchange Capability for the Portable LED Phototherapy Skin Treatment Device. In the invented device, The LED based phototherapy device is designed to be capable of changing heads with different wavelength LED combinations (as shown in FIG. 4). With the current design, the LED array head can be replaced easily for treating different skin conditions.

In a test, a handhold device for treatment of the skin based on Light Emitting Diode (LED) assisted by vacuum and negative ions uses: The wavelength of the light ranges from 400 to 880 nm according to the treatment of the skin; The exchangeable LED head in the device is used to quickly exchange the head with different wavelengths and/or their combinations; The intensity of device output light can be adjusted up to 20-50 mW/cm² upon number of turned on LEDs A build-in MEMS or other mini vacuum pump supplies negative pressure (125 mmHg) in the chamber; A build-in mini negative ion generator supplies negative ion with high density (>1 million negative ions/cm³) in the chamber; and A build-in mini 3-way solenoid valve controls mode of vacuum or negative ion in the chamber.

While some embodiments of the invention have been described by way of illustration, it will be apparent that the invention can be carried into practice with many modifications, variations and adaptations, and with the use of numerous equivalents or alternative solutions that are within the scope of persons skilled in the art, without departing from the spirit of the invention or exceeding the scope of the claims.

What is claimed is:

1. An apparatus for skin treatments, comprising: a) a chamber placed on a skin target which is formed with an aperture on the distal end thereof b) means for supplying negative ion into said chamber, therefore the ion can be used to treat the skin target.

2. The apparatus according to claim 1, further comprising means for controlling the ion density by controlling said means for supplying ion.

3. The apparatus according to claim 1, further comprising at least one light source with one wavelength or multi-wavelength, wherein the light source is arranged inside the chamber to provide optical energy in the predetermined wavelength(s) through said aperture to the skin target.

4. The apparatus according to claim 3, wherein said chamber comprises a vacuum chamber, the level of the applied vacuum suitable for drawing said skin target to said vacuum chamber via said aperture; and the applied vacuum is controlled by a vacuum controlling means for adjusting the pressure of said chamber.

5. The apparatus according to claim 4, wherein said vacuum controlling means modulate pressure/vacuum of said chamber to obtain a predetermined optical energy absorb in a predetermined depth below the skin target.

6. The apparatus according to claim 3, wherein the light source is a LED array head, said LED array comprises at least one LED, said LED array head is separated from the adjoining skin surface by a gap ranging from 0.1 to 50 mm.

7. The apparatus according to claim 4, wherein said vacuum chamber is applied by a vacuum pump.

8. The apparatus according to claim 7, wherein said vacuum pump is a miniature vacuum pump to form a micro electro mechanical system for applied the vacuum.

9. The apparatus according to claim 7, wherein the applied vacuum is modulated by at least one control valve.

10. The apparatus according to claim 4, further comprising control means for controlling the at least one operation of: said supplying ion; said light source;
said applied vacuum; said modulation of the applied vacuum.

11. The apparatus according to claim 6, wherein the LED comprises at least two LEDs with different wavelength, the control means further comprises LED control means to control each LED's power on or off upon the treatment requirement.

12. The apparatus according to claim 7, wherein the LED array is a LED array head comprising at least two kind of LED array head with different LED, respectively, said at least kind of LED array head are exchangeable upon the requirement.

13. The apparatus according to claim 1, wherein said means for supplying ion into said chamber comprises an ion generator for generating ion in the air; and a pump for pumping air with the ion into the chamber via a port.

14. The apparatus according to claim 13, further comprising a valve for controlling the flow capacity of the ion air pumped into the chamber; and means for controlling ion density, coupled to said valve and said pump.

15. The apparatus according to claim 13, wherein said ion generator comprises a voltage up converter for converting the DC voltage to a predetermined voltage, said predetermined voltage is coupled to a discharge port for generating said ion.

16. An apparatus for skin treatments, comprising: a) a chamber placed on a skin target which is formed with an aperture on the distal end thereof b) means for applying vacuum to said chamber, therefore the skin target is able to be treated upon the applied vacuum; and c) at least one light source with one wavelength or multi-wavelength, wherein the light source is arranged inside the chamber to provide optical energy in the predetermined wavelength(s) through the aperture to the skin target.

17. The apparatus according to claim 16, wherein the light source is a LED array head, said LED array comprises at least one LED, said LED array head is separated from the adjoining skin surface by a gap ranging from 0.1 to 50 mm.

18. The apparatus according to claim 17, wherein the means for applying vacuum comprises a miniature vacuum pump to form a micro electro mechanical system for applying the vacuum to the chamber; and the applied vacuum is modulated by at least one control valve; and the apparatus further comprising control means for controlling the at least one operation of: said light source;
said applied vacuum; said modulation of the applied vacuum.

19. An apparatus for skin treatments, comprising: a) a chamber placed on a skin target which is formed with an aperture on the distal end thereof b) means for supplying negative ion into said chamber, therefore the ion can be used to treat the skin target and c) at least one light source with one wavelength or multi-wavelength, wherein the light source is arranged inside the chamber to provide optical energy in the predetermined wavelength(s) through the aperture to the skin target.

20. The apparatus according to claim 19, wherein the means for supplying ion comprises a miniature vacuum pump to form a micro electro mechanical system for applying the ion to the chamber; and the applied ion density is modulated by at least one control valve; the apparatus further comprising a control means for controlling the at least one operation of: said light source;
said supplied ion; said modulation of the density of the supplied ion.
